(19) United States
(12) Patent Application Publication
(10) Pub. No.: US 2025/0290165 A1
(43) Pub. Date: Sep. 18, 2025

(54) VIRAL VARIANT DETECTION

(71) Applicants: Transformative Biotech, LLC, Boulder, CO (US); The Regents of The University of Colorado, Aurora, CO (US)

(72) Inventors: Shi-Long Lu, Englewood, CO (US); Brian L. Harry, Denver, CO (US); Yue Qiu, Englewood, CO (US); Eva Yao, Aurora, CO (US); Robert E. Blomquist, Boulder, CO (US)

(21) Appl. No.: 18/559,120

(22) PCT Filed: Jun. 24, 2022
(86) PCT No.: PCT/US22/34919

§ 371 Date: Nov. 6, 2023

Related U.S. Application Data
(60) Provisional application No. 63/185,015, filed on May 6, 2021.

(51) Int. Cl. C12Q 1/70; C12Q 1/686

(52) U.S. Cl. CPC C12Q 1/701; C12Q 1/686

(57) ABSTRACT

The invention provides compositions and methods allowing for rapid detection of SARS-CoV-2 variants.

### CROSS-REFERENCE TO RELATED APPLICATIONS

This application claims priority to and the benefit of U.S. Provisional Application No. 63/185,015, filed May 6, 2021, the content of which is incorporated herein in its entirety.

### TECHNICAL FIELD

The invention generally relates to diagnostic methods, and, more particularly, to compositions and methods for rapid PCR detection of SARS-CoV-2 variants.

### BACKGROUND

The rapid global spread of contagious diseases presents a major healthcare challenge. For example, the rapid spread of the severe acute respiratory syndrome coronavirus-2 (SARS-CoV-2), resulting in a global pandemic, has placed an emphasis on the criticality of rapid and early detection. As the pandemic has progressed, new, more infectious variants have spread. These variants can cause more severe disease with poorer prognosis and, importantly, respond differently to treatment. Accordingly, rapid early detection and now variant identification are critical to successful treatment.

Conventional detection techniques for many infectious diseases involve the use of polymerase chain reaction (PCR). PCR is a technique used to selectively amplify a specific region of DNA of interest (the DNA target). For example, various real-time PCR assays (also referred to as quantitative PCR (qPCR)) for detecting SARS-CoV-2 RNA have been developed worldwide, with different targeted viral genes or regions. However, those assays may not detect some of the variants of concern or, if they do, may not be able to differentiate between wild type and the variants of concern. Diagnoses may then be missed allowing the variants to spread further and, if detected, correct treatment may be delayed due to misidentification as wild type infection.

Another drawback of current qPCR detection approaches is that they rely on an initial step of isolating and purifying nucleic acids from a clinical sample as part of the viral testing protocol. For example, the application of qPCR for the relative quantification of an RNA typically requires: (1) the isolation and purification of total RNA from the sample; (2) elution and possible concentration of the material; and (3) the use of purified RNA in a reverse-transcription (RT) reaction resulting in complementary DNA (cDNA), which is then utilized for the qPCR reaction.

The initial nucleic acid isolation and purification step (i.e., extraction step) required in conventional methods, prior to undergoing PCR, constitutes a major bottleneck in the diagnostic process, as it remains both manually laborious and expensive, and further increases the chances of accidental contamination and human error. Furthermore, in a period of high demand, a shortage of nucleic acid extraction supplies can exacerbate the limitations of such viral detection methods. Accordingly, improved methods for accurate, specific, and rapid diagnosis of pathogens is needed.

### SUMMARY

The present invention provides compositions and methods for rapid PCR detection and differentiation of known SARS-CoV-2 variants. For example, using variant-specific primers, methods and compositions disclosed herein detect and differentiate between the B.1.1.7 (UK), P.1 (Brazil and Japan), B.1.351 (South Africa), and B.1.429/427 (California) variants (referred herein collectively as Variants of Concern (VOC)). Variants may be more infectious, cause more severe disease, and require different treatment than wild type SARS-CoV-2 infections. Accordingly, by providing rapid PCR identification of variants, compositions and methods of the invention allow for the correct treatment to be delivered quickly without the need for time-consuming and expensive sequencing.

In certain embodiments, extraction-free detection and analysis techniques are used with variant-specific primers of the invention. Those techniques include compositions for processing a biological sample and providing usable nucleic acid for subsequent amplification and/or detection, while eliminating the need for an initial nucleic acid extraction step. Compositions of the present invention may include, for example, a unique buffer composition for sample transport and preparation that, when mixed with a sample of interest, is capable of preparing nucleic acid from the sample that is suitable for direct nucleic acid amplification and analysis without the need for initial nucleic acid extraction (i.e., isolation and purification of the nucleic acid). In one aspect, the invention avoids conventional approaches for viral detection, which include an RNA extraction step using industrial RNA extraction kits and techniques. Instead, according to the invention, sample testing is direct and avoids the extraction step.

In a preferred embodiment, compositions and methods of the present invention improve upon conventional viral testing and detection approaches by reducing the number of steps required for sample preparation and testing and avoiding the need for time consuming and costly sequencing. In turn, the time required for detecting VOCs and adjusting treatment accordingly is greatly reduced, improving patient prognosis.

Certain aspects of the invention include methods for detecting SARS-CoV-2 variants. Preferred methods include providing a biological sample comprising nucleic acid, amplifying the nucleic acid with primers specific to one or more target SARS-CoV-2 variants without amplifying wild type SARS-CoV-2 nucleic acid, and analyzing amplicons produced in the amplifying step to detect the presence of the one or more target SARS-CoV-2 variants.

In certain embodiments, methods further comprise detecting the presence of a SARS-CoV-2 infection by, prior to the amplifying step, using primers that amplify one or more target SARS-CoV-2 variants and wild type SARS-CoV-2. The one or more target SARS-CoV-2 variants may be selected from the group consisting of B.1.1.7, P.1, B.1.351, B.1.429/427, and other variants of SARS-CoV-2. The primers may be specific to two or more target SARS-CoV-2 variants. In certain embodiments, the primers may be specific to three or more target SARS-CoV-2 variants.

The three or more target SARS-CoV-2 variants may include B.1.1.7, P.1, and B.1.351. In one example, the primers target an ORF-1A region. In certain embodiments, the primers target a deletion at position 3675-35677 of the ORF-1A region relative to wild type SARS-CoV-2. Exemplary primers comprise SEQ ID NO: 1 and SEQ ID NO: 2. In some embodiments, the amplifying step comprises quantitative PCR (qPCR) using a probe comprising SEQ ID NO: 3.

In certain embodiments, the target SARS-CoV-2 variants comprise B.1.1.7 and the primers target an S region. The primers may target a deletion at position 69-70 of the S region relative to wild type SARS-CoV-2. Exemplary primers comprise SEQ ID NO: 4 and SEQ ID NO: 5. The amplifying step may comprise quantitative PCR (qPCR) using a probe comprising SEQ ID NO: 6.

In various embodiments, the target SARS-CoV-2 variants comprise B.1.351 and the primers target a G25563T or G28887T substitution relative to wild type SARS-CoV-2. Exemplary primers comprise SEQ ID NO: 19 and SEQ ID NO: 20 or SEQ ID NO: 22 and SEQ ID NO: 23. In this example, the amplifying step comprise quantitative PCR (qPCR) using a probe comprising SEQ ID NO: 21 or SEQ ID NO: 24.

In some embodiments, target SARS-CoV-2 variants comprise B.1.429/427 and the primers target G27890T/G27987T or G28191T/A28272T substitutions relative to wild type SARS-CoV-2. Exemplary primers comprise SEQ ID NO: 25 and SEQ ID NO: 26 or SEQ ID NO: 28 and SEQ ID NO: 29. In this example, the amplifying step comprises quantitative PCR (qPCR) using a probe comprising SEQ ID NO: 27 or SEQ ID NO: 30.

In certain embodiments, methods further comprise mixing the biological sample in a buffer composition comprising nuclease-free water, an antifungal, an antibiotic, a ribonuclease inhibitor, and a reducing agent. The amplifying step is performed on the nucleic acid in the buffer without prior extraction of the nucleic acid. The biological sample may be any bodily fluid. The body fluid is selected from the group consisting of saliva, sputum, mucus, phlegm, and urine. In certain embodiments, the reducing agent is a Tris(2-carboxyethyl) phosphine hydrochloride solution. An exemplary antifungal comprises an Amphotericin B and an exemplary antibiotic comprise Penicillin Streptomycin. The biological sample may be obtained via a nasal or throat swab. Methods may further comprise the step of heat-inactivating the biological sample mixed with the buffer composition prior to said amplifying step. The mixture of biological sample and buffer composition can be heated to about 95° C. for about 5 minutes.

In some embodiments, the biological sample is obtained from a subject suspected of having a SARS-CoV-2 infection, and methods further comprise selecting a treatment regimen based on the detected presence of the one or more target SARS-CoV-2 variants. The treatment regimen may be different than the recommended treatment regimen for a wild type SARS-CoV-2 infection.

Methods of the invention utilize a primer set that selectively amplifies nucleic acid from one or more target SARS-CoV-2 variants without amplifying nucleic acid from wild type SARS-CoV-2. In certain aspects, primer sets of the invention are arranged in an array for a multiplex analysis for simultaneous detection of SARS-CoV-2 infections and identification of infection type among a set of wild type and known variants. In various embodiments, the array may include a plurality of wells each comprising a different primer-probe set for qPCR detection of one or more specific variant in a single biological sample.

### BRIEF DESCRIPTION OF THE DRAWINGS

FIG. 1 lists target regions in different SARS-CoV-2 variants for variant-specific detection and primer-probe sets targeting the listed variants.

FIG. 2 maps mutation targets for various VOCs.

FIG. 3 shows results of rapid PCR detection of both wild type and VOC variants using a single primer set targeting the N1 region.

FIG. 4 shows a map of common mutations in the SARS-CoV-2 genome.

FIG. 5 illustrates Cq values for primer-probe sets targeting four different commonly mutated regions of the SARS-CoV-2 genome when exposed to a B.1.1.7 positive control.

FIG. 6 illustrates Cq values for primer-probe sets targeting four different commonly mutated regions of the SARS-CoV-2 genome when exposed to a wild type positive control.

FIG. 7 shows comparative Cq values of N region mutation targeting primers for wild type and B.1.1.7 positive controls.

FIG. 8 shows comparative Cq values of Orf1a region mutation targeting primers for wild type and B.1.1.7 positive controls.

FIG. 9 shows comparative Cq values of a first set of S region mutation targeting primers for wild type and B.1.1.7 positive controls.

FIG. 10 shows comparative Cq values of a second set of S region mutation targeting primers for wild type and B.1.1.7 positive controls.

FIG. 11 shows results of NGS validation of rapid PCR detection of SARS-CoV-2 variants using methods and compositions of the invention.

FIG. 12 shows an exemplary flow chart for determining specific VOC infections in a sample.

### DETAILED DESCRIPTION

There is increasing concern about the spread of contagious diseases, such as influenza, common colds, potentially lethal viruses, or microbial or viral diseases, both known and unknown. In 2019 an outbreak of SARS-CoV-2 resulted in a global pandemic and outbreak of respiratory illness, resulting in numerous deaths. Subsequently several variants of concern developed, characterized by increased rates of transmission, more severe disease and poorer prognoses, and different treatment responses. These variants have driven new waves of infection and caused concern regarding their detectability using current tests and their responsiveness to current vaccines. Furthermore, as these variants may respond differently to treatment and cause more severe disease, there is a pressing need to quickly and cheaply identify not just SARS-CoV-2 infections but whether the infection is with one of the VOCs. Additionally, tracking the spread of the different variants is helpful in understanding and overcoming a pandemic and cheap rapid variant testing provides a valuable tool for doing so.

The present invention provides compositions and methods allowing for rapid detection of SARS-CoV-2 variants, including by extraction-free, direct PCR techniques. More specifically, the invention provides compositions including primer sets for selectively amplifying one or more VOCs without amplifying wild type virus, thereby allowing rapid diagnosis and identification of variants without the need for time and cost intensive sequencing. In various embodiments, workflows and primer-probe sets are disclosed for rapid identification of which of the four presently known VOCs is present in a biological sample. In certain embodiments, different sets of primers can be included in an array or multiplexed analysis such that multiple identification tests can be run simultaneously on a single sample. Methods of the invention provide a point-of-care solution for rapid and accurate identification of pathogens even if a laboratory is not available.

In certain embodiments, methods are provided for processing a biological sample and providing usable DNA for subsequent PCR assays, while eliminating the need for an initial RNA extraction step. Methods of the invention may use a unique buffer composition for sample transport and preparation that, when mixed with a sample of interest, is capable of preparing nucleic acid from the sample that can be used directly for nucleic acid amplification and analysis without the need for initial nucleic acid extraction (i.e., isolation and purification of the nucleic acid). After clinical samples are provided in the unique buffer composition, viral particles may be inactivated either through heating or by direct lysis in the buffer. The inactivated samples can then be used for downstream qPCR diagnostic testing using VOC-specific primer-probe sets of the invention.

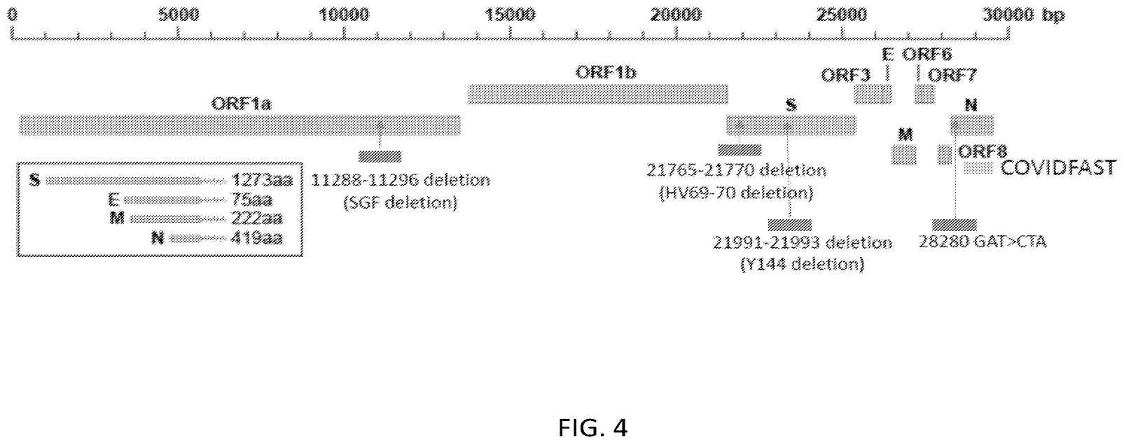

Rapid replication and large world-wide infection rates have led to the emergence of several SARS-CoV-2 variants including the aforementioned VOCs that exhibit concerning transmission or infection characteristics that differ from the wild type virus. Some commonly observed mutations are mapped in FIG. 4.

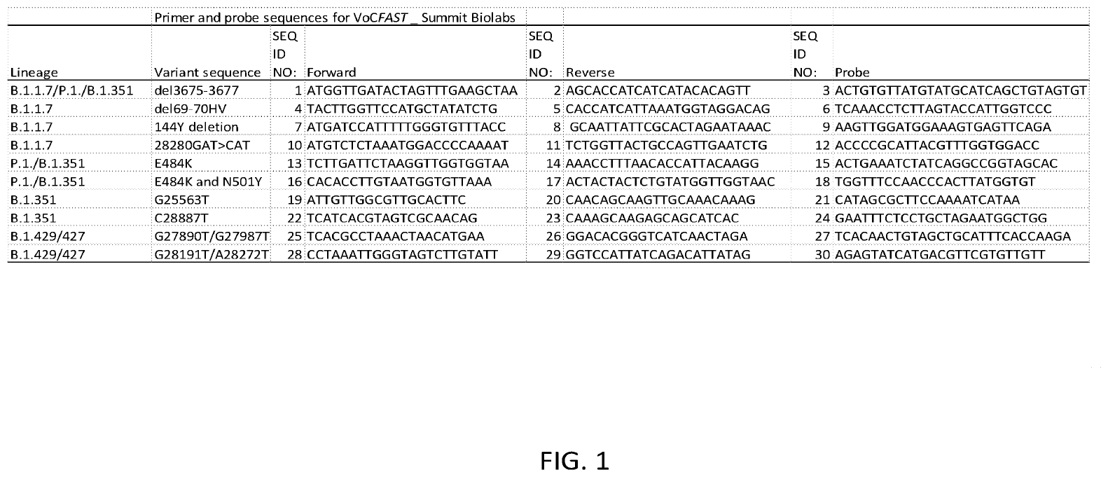

Exemplary primer sets are listed in FIG. 1 including the VOC(s) they selectively amplify/detect, the variant sequence they target, and the forward primer, reverse primer, and probe sequence.

In various embodiments, B.1.1.7, P.1, B.1.351, and other variants may be jointly detected using primer-probe sets targeting a deletion at the 3675-3677 position relative to the wild type SARS-CoV-2 virus. Exemplary primers targeting that mutation are provided in FIG. 1 with SEQ ID NO: 1 (forward) and SEQ ID NO: 2 (reverse) along with a probe comprising SEQ ID NO: 3. Because the treatment methods for these VOCs are similar, in the interest of rapid response, additional differentiation may not be required or may be postponed. Accordingly, the determination that a patient has one of those three VOCs using a rapid qPCR test as described herein can provide valuable time sensitive information for treating the patient while minimizing the time and costs associated with multiplex testing or sequencing. The skilled artisan understands that the invention is easily applicable to arising variants, in that the invention is based, in part, on the preferential amplification of variants over the standard type viral sequence. All that one needs to apply the invention to a new variant is the identification of sequences that select for a discovered variant.

In certain embodiments, a B.1.1.7 variant is detected using primer-probe sets targeting a deletion at the 69-70 position relative to the wild type SARS-CoV-2 virus. Exemplary primers targeting that mutation are provided in FIG. 1 with SEQ ID NO: 4 (forward) and SEQ ID NO: 5 (reverse) along with a probe comprising SEQ ID NO: 6.

In certain embodiments, a B.1.1.7 variant is detected using primer-probe sets targeting a 144Y deletion relative to the wild type SARS-CoV-2 virus. Exemplary primers targeting that mutation are provided in FIG. 1 with SEQ ID NO: 7 (forward) and SEQ ID NO: 8 (reverse) along with a probe comprising SEQ ID NO: 9.

In certain embodiments, a B.1.1.7 variant is detected using primer-probe sets targeting a GAT>CAT mutation at the 28280 position relative to the wild type SARS-CoV-2 virus. Exemplary primers targeting that mutation are provided in FIG. 1 with SEQ ID NO: 10 (forward) and SEQ ID NO: 11 (reverse) along with a probe comprising SEQ ID NO: 12.

In certain embodiments, P.1 and B.1.351 variants are jointly detected using primer-probe sets targeting an E484K substitution relative to the wild type SARS-CoV-2 virus. Exemplary primers targeting that mutation are provided in FIG. 1 with SEQ ID NO: 13 (forward) and SEQ ID NO: 14 (reverse) along with a probe comprising SEQ ID NO: 15.

In certain embodiments, P.1 and B.1.351 variants are jointly detected using primer-probe sets targeting E484K and N591Y substitutions relative to the wild type SARS-CoV-2 virus. Exemplary primers targeting that mutation are provided in FIG. 1 with SEQ ID NO: 16 (forward) and SEQ ID NO: 17 (reverse) along with a probe comprising SEQ ID NO: 18.

In certain embodiments, a B.1.351 variant are detected using primer-probe sets targeting a G25563T substitution relative to the wild type SARS-CoV-2 virus. Exemplary primers targeting that mutation are provided in FIG. 1 with SEQ ID NO: 19 (forward) and SEQ ID NO: 20 (reverse) along with a probe comprising SEQ ID NO: 21.

In certain embodiments, a B.1.351 variant are detected using primer-probe sets targeting a C28887T substitution relative to the wild type SARS-CoV-2 virus. Exemplary primers targeting that mutation are provided in FIG. 1 with SEQ ID NO: 22 (forward) and SEQ ID NO: 23 (reverse) along with a probe comprising SEQ ID NO: 24.

In certain embodiments, a B.1.429/427 variant are detected using primer-probe sets targeting G27890T and G27987T substitutions relative to the wild type SARS-CoV-2 virus. Exemplary primers targeting that mutation are provided in FIG. 1 with SEQ ID NO: 25 (forward) and SEQ ID NO: 26 (reverse) along with a probe comprising SEQ ID NO: 27.

In certain embodiments, a B.1.429/427 variant are detected using primer-probe sets targeting G28191T and A28272T substitutions relative to the wild type SARS-CoV-2 virus. Exemplary primers targeting that mutation are provided in FIG. 1 with SEQ ID NO: 28 (forward) and SEQ ID NO: 29 (reverse) along with a probe comprising SEQ ID NO: 30.

In certain embodiments, a B.1.429/427 variant are detected using primer-probe sets targeting L452R (T22917G) substitutions relative to the wild type SARS-CoV-2 virus. Exemplary primers targeting that mutation can comprise TGGTAATTATAATTACCGGT (forward SEQ ID NO: 31), ACTGAAATCTATCAGGCCGGTAGCAC (reverse SEQ ID NO: 32) along with a probe comprising AAACCTTCAACACCATTACAAGG (SEQ ID NO: 33).

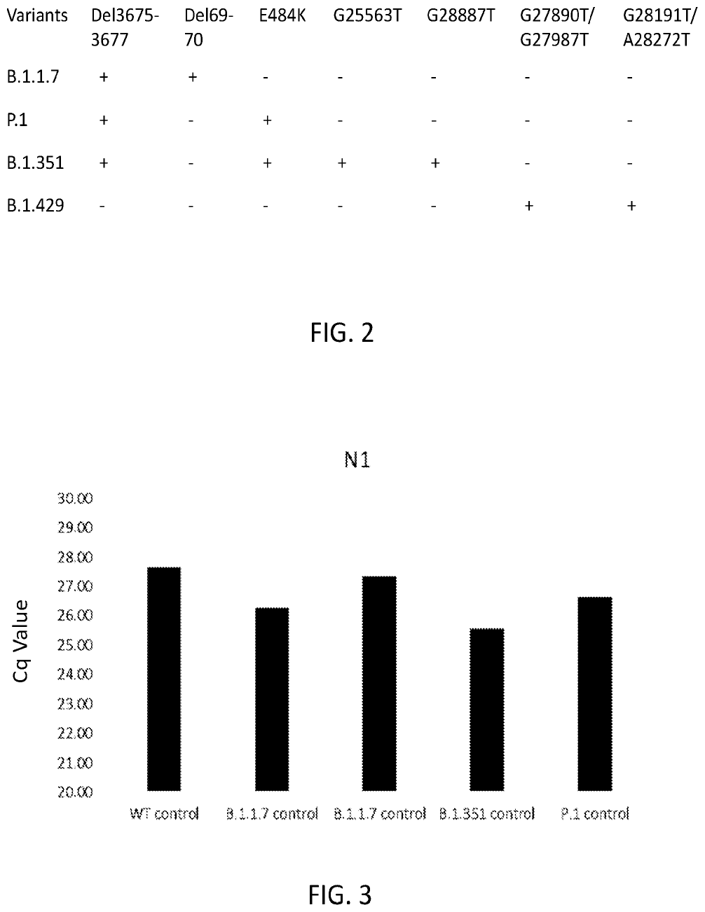

The aforementioned mutations are mapped onto each of the 4 VOCs in FIG. 2 indicating which target mutations would generate positive or negative results for which VOC(s).

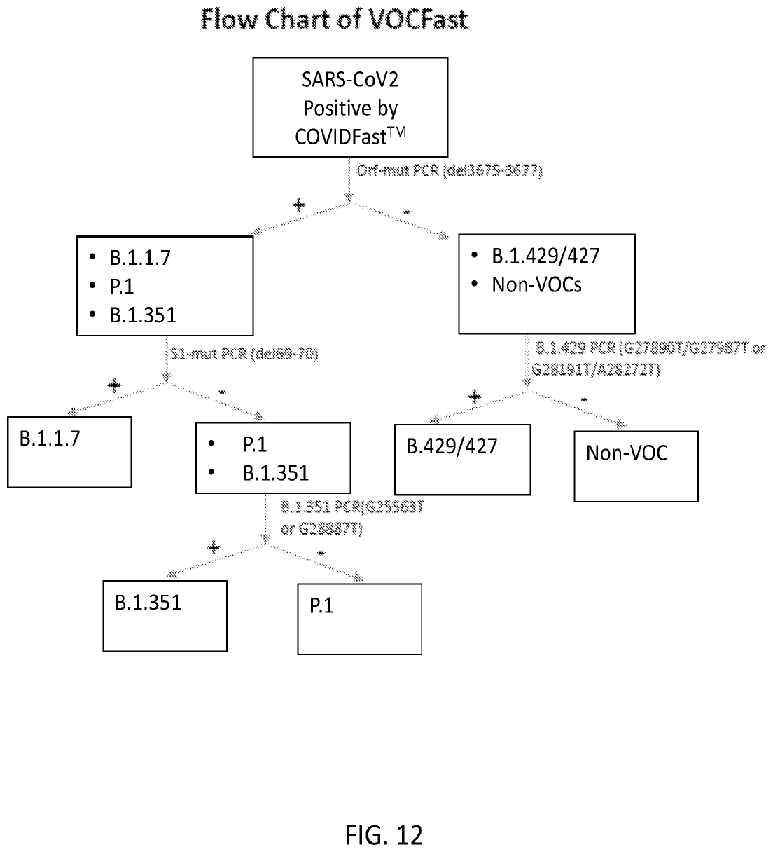

Upon detection of variants B.1.1.7, P.1, or B.1.351 using the Orf-mut primers as described above (e.g., SEQ ID NOS: 1 and 2) or any other primers that jointly detect more than one variant, it may then be desirable to further differentiate between the detected variants. Accordingly, a sample may be subjected to a workflow as shown in FIG. 12. A sample may be initially tested with N1 primers or any other means of detecting general SARS-CoV-2. In order to determine if the infection if wild type SARS-CoV-2 or a VOC, the sample can then be tested using Orf-mut primers as discussed above. A positive result would then indicate that the presence of one of the B.1.1.7, P.1, or B.1.351 variants while a negative result would indicate a wild type or non-VOC variant or infection with the B.1.429/427 variant.

Upon a positive Orf-mut result, the sample can be tested with primers targeting the S region (e.g., S1-mut primers targeting del69-70). A positive result there indicates that the infection is the B.1.1.7 variant. If a negative result is found, the sample can then be tested with B.1.351 specific primers as listed above where a positive result indicates the infection is with the B.1.351 variant and a negative result indicates the infection is P.1.

If a negative Orf-mut result is obtained, the sample can be tested with B.1.429/427-specific primers as listed above with a positive result indicating infection with that variant and a negative result indicating a wild type infection or other non-VOC variant. Accordingly, by running through the exemplary flow chart, a specific VOC infection can be identified in a maximum of three rapid qPCR reactions. In various embodiments, steps of the aforementioned flow chart can be carried out simultaneously using multiplex analysis. For example, a multi-well plate or array may be used wherein sample can be introduced to different primer sets in different wells or different locations such that all test results for a given sample can be determined at once. Alternatively, each well or location could contain a primer set specific to only one of the four VOCs. In certain embodiments, a single reaction mixture can be prepared using the Orf-mut primers as well as B.1.429/427-specific primers wherein any positive result would be indicative of a VOC infection.

In general, the workflow for qPCR detection using primer/probe sets of the invention comprises obtaining a biological sample from an individual suspected of being infected. The method of sample collection, as well as the type of sample collected, may vary. For example, the biological sample may include a body fluid and may be collected in any clinically-acceptable manner. The fluid sample is generally collected from a patient either exhibiting signs or symptoms of the disease or suspected of having contracted the disease due to interaction with others that have tested positive for the disease. The methods described above are applicable to the detection of any discovered variant or combination of variants.

A body fluid may be any liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CS. A sample also may be media containing cells or biological material. A sample may also be a blood clot, for example, a blood clot that has been obtained from whole blood after the serum has been removed. In certain embodiments, the sample is blood, saliva, or semen collected from the subject.

For SARS-CoV-2, a biological sample is generally collected via a nasopharyngeal or throat swab, or, in some cases, the sample may be saliva. Next, the sample is prepared for subsequent analysis. Preparation of the sample can include mixing the sample with a buffer composition capable of preparing nucleic acid from the biological sample suitable for nucleic acid amplification without initial extraction of the nucleic acid.

As previously noted, many viral testing approaches rely on an initial step of isolating and purifying nucleic acids from a clinical sample as part of the viral testing protocol. For example, the application of qPCR for the relative quantification of an RNA of interest is preceded by: (1) the isolation and purification of total RNA from the sample; (2) elution and possible concentration of the material; and (3) the use of purified RNA in a reverse-transcription (RT) reaction resulting in complementary DNA (cDNA), which is then utilized for the qPCR reaction. Primer-probe sets of the invention can be used on RNA prepared using such methods. However, the initial nucleic acid isolation and purification step (i.e., extraction step) required in current methods, prior to undergoing PCR, constitutes a major bottleneck in the diagnostic process, as it remains both manually laborious and expensive, and further increases the chances of accidental contamination and human error.

Accordingly, in preferred embodiments, the biological sample can be processed to provide usable DNA for subsequent PCR assays while eliminating the need for an initial RNA extraction step. For example, a unique buffer composition can be used for sample preparation such that, when mixed with the biological sample, it is capable of preparing nucleic acid from the sample which is able to be being directly used for nucleic acid amplication and analysis without the need for initial nucleic acid extraction (i.e., isolation and purification of the nucleic acid).

Exemplary primers and probes for the detection of wild type SARS-CoV-2 have been disclosed by the Chinese CDC (targeting the N and ORF1ab genes) and the WHO (targeting the E gene) and are provided in Tao S, et al., 2020 and Dong, I et al. 2020. Compositions and methods of the invention for the detection of wild type COVID-19 infection using ddPCR of saliva and nasopharyngeal samples contemplate using the same primers and probes discussed therein. Furthermore, in some embodiments, the step of performing the one or more PCR assays includes using a primer-probe set specific to ribonuclease P (RNP).

An exemplary method for an extraction-free, real-time RT-qPCR test intended for the qualitative detection of nucleic acid from SARS-CoV-2 VOCs in biological specimens (spit or swab samples) collected and processed via unique buffer compositions of the present invention is described below. In the case of a nasopharyngeal swab, the swab is used for the collection of respiratory mucosa and then placed within an acceptable vessel which includes a unique buffer composition of the present invention, used as both a transport medium and sample preparation medium for the potential SARS-CoV-2 viral particles. In the case of saliva, a patient will simply spit in an acceptable vessel, at which point the saliva will then be transferred to another vessel containing the unique buffer composition for sample preparation. Upon being collected and provided within the unique buffer composition, viral particles may be inactivated either through heating or by direct lysis in the buffer. The inactivated samples can then be used for downstream qPCR diagnostic testing without the need for the additional RNA extraction step (isolation and purification) that conventional approaches rely on. Rather, the prepared sample may be transferred to a PCR-plate (96/384-well) format in which cDNA synthesis by RT and detection by qPCR may take place. Accordingly, unlike the widely used approach, which includes an RNA extraction step using industrial RNA extraction kits, direct sample testing circumvents this process by omitting extraction.

### EXAMPLES

### Example 1—Comparisons of Mutant Primer/Probe Sets in Detecting Wildtype and B.1.1.7 Mutant Controls

Primer sets were developed targeting various regions mutated in the VOCs. General SARS-CoV-2 primers targeting the N1 region (e.g., primers designated COVIDFAST in the mapping in FIG. 4) were used for qPCR testing against wild type and VOC controls (except for B.1.429/427) and found provide detectable signal for each of the wild type and B.1.1.7, B.1.351, and P.1 variants as illustrated in FIG. 3.

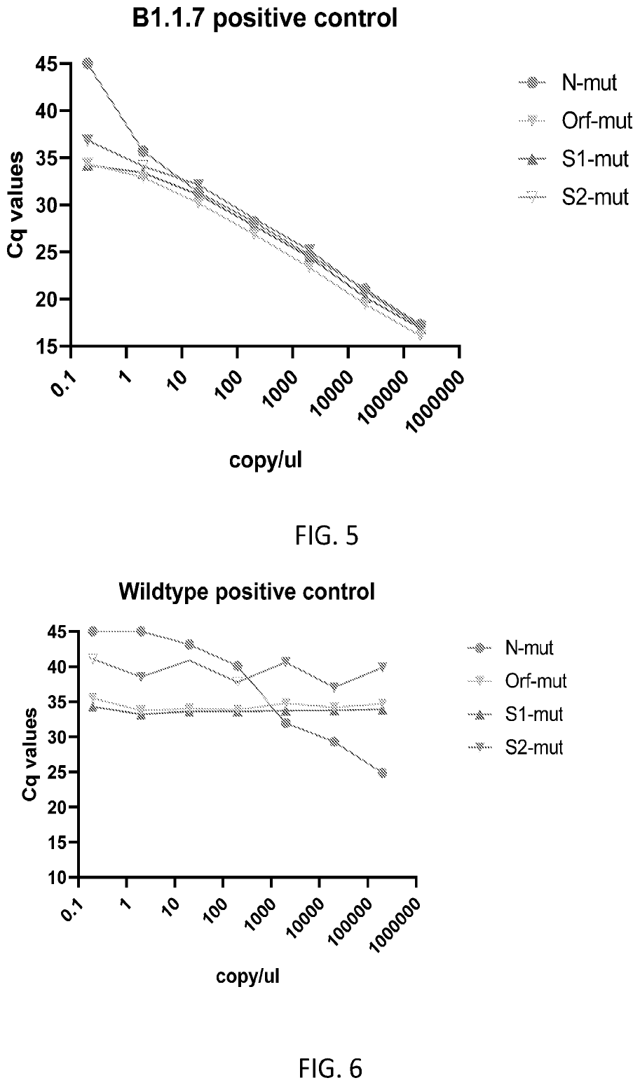

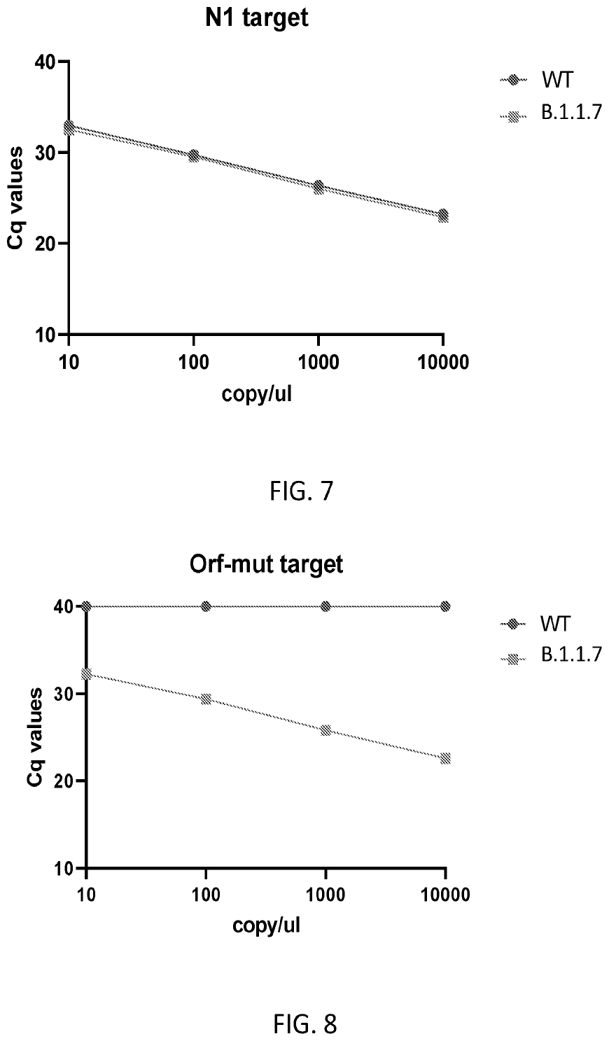

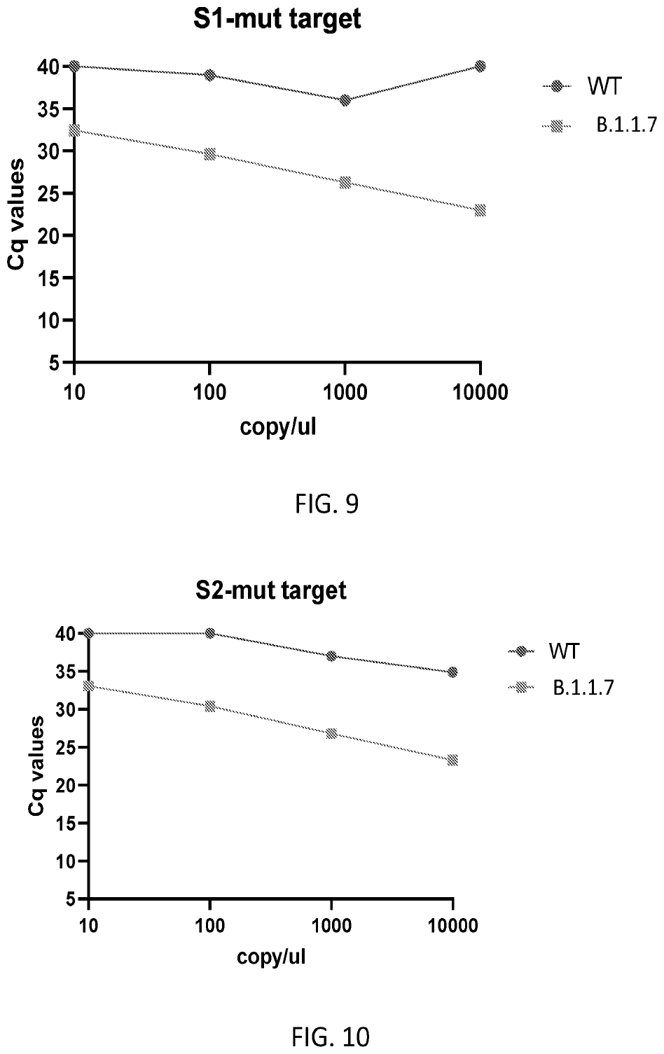

In order to distinguish between the wildtype and VOCs, primers targeting some of the commonly mutated regions shown in FIG. 4 were designed. For example, four sets of primers directed to the ORF1a, S, and N regions were designed in an attempt to distinguish between B.1.1.7 and wild type SARS-CoV-2. Proxy PCR was performed using both synthetic positive controls (Twist, control 14, and 15). FIGS. 5-10 illustrate the effectiveness of those four target regions for differentiating between wild type and the B.1.1.7 variant. FIGS. 5-8 shows quantification cycle (Cq) values required to produce signal using each of the four primer sets at various concentrations of virus. As shown in FIG. 5, each of the four primer sets (directed to mutations in the N, ORF1a, and S regions) successfully detected the B.1.1.7 positive control. As shown in FIG. 6, however, the N region mutation-specific primers also responded to the wild type positive control. FIG. 7 further illustrates that the N region mutation primers did not produce differential signals in the presence of wild type and B.1.1.7. Accordingly, primers targeting the N region mutation would not be successful in differentiating between wild type and VOC infections. However, as shown in FIG. 8 (Orf-mut targeting primers), FIG. 9 (S1-mut targeting primers), and FIG. 10 (S2-mut targeting primers) all produced differential signals for wild type and B.1.1.7. Of those three, the Orf-mut targeting primers provided the largest contrast in responsiveness for detecting B.1.1.7 variants.

### Example 2—ORF1a Variant Primer Validation

Table 1 shows PCR testing results for 59 positive COVID19 samples from Nov. 12, 2020 through Feb. 18, 2021 (Sample Nos. 1-17 from Nov. 12, 2020 to Jan. 10, 2021; Sample Nos. 18-42 from Jan. 18, 2021; and Sample Nos. 43-59 from Feb. 12, 2021 to Feb. 18, 2021). Among the tested samples, no VOCs (B.1.429/427 no included) were detected. These results are in line with the prevalence (or lack thereof) of the tested variants in the general population at those times. The samples were tested using wild type primer sets targeting the N1 region and a variant specific primer set targeting the variant specific (B.1.1.7, P.1, and B.1.351) mutations to the ORF1a region.

TABLE 1

| | | WT-primer | Mutant-primer |
|---|---|---|---|
| Sample # | Pts ID# | (N1) | (Orf-mut) |
| 1 | STF35 | 18.45 | 38.48 |
| 2 | STF47 | 26.24 | 41.91 |
| 3 | STF56 | 26.85 | 39.89 |
| 4 | STF62 | 21.89 | NaN |
| 5 | STF64 | 30.12 | 41.05 |
| 6 | STF65 | 22.35 | 39.96 |
| 7 | STF68 | 34.55 | 42.38 |
| 8 | STF69 | 33.12 | 37.86 |
| 9 | STF70 | 28.92 | 43.42 |
| 10 | STF73 | 30.45 | 40.95 |
| 11 | STF75 | 22.58 | 42.36 |
| 12 | STF77 | 23.00 | 42.18 |
| 13 | STF78 | 20.60 | 41.65 |
| 14 | STF80 | 30.77 | 38.29 |
| 15 | STF84 | 32.30 | 38.14 |
| 16 | STF89 | 22.10 | NaN |
| 17 | STF90 | 23.00 | 38.40 |
| 18 | CCC-1 | 20.50 | 41.72 |
| 19 | CCC-2 | 31.57 | 44.14 |
| 20 | CCC-3 | 17.87 | 35.68 |
| 21 | CCC-4 | 19.59 | 44.22 |
| 22 | CCC-5 | 27.42 | NaN |
| 23 | CCC-6 | 21.96 | NaN |
| 24 | CCC-7 | 26.54 | 37.03 |
| 25 | CCC-8 | 21.23 | NaN |
| 26 | CCC-9 | 26.81 | 39.88 |
| 27 | CCC-10 | 28.10 | 42.72 |
| 28 | CCC-11 | 23.40 | NaN |
| 29 | CCC-12 | 40.22 | 39.27 |
| 30 | CCC-13 | 24.33 | NaN |
| 31 | CCC-14 | 21.49 | NaN |
| 32 | CCC-15 | 23.69 | 42.21 |
| 33 | CCC-16 | 17.72 | NaN |
| 34 | CCC-17 | 28.94 | 38.41 |
| 35 | CCC-18 | 24.19 | NaN |
| 36 | CCC-19 | 23.97 | 39.77 |
| 37 | CCC-20 | 26.34 | 42.78 |
| 38 | CCC-21 | 21.23 | 43.43 |
| 39 | CCC-22 | 32.65 | NaN |
| 40 | CCC-23 | 18.49 | NaN |
| 41 | CCC-24 | 28.48 | 44.08 |
| 42 | CCC-25 | 27.11 | NaN |
| 43 | R1-C5 | 26.20 | NaN |
| 44 | R2-G1 | 27.12 | NaN |
| 45 | R4-A6 | 34.39 | NaN |
| 46 | R4-B5 | 28.53 | NaN |
| 47 | W-4 | 24.89 | NaN |
| 48 | W-6 | 23.21 | NaN |
| 49 | W-10 | 24.18 | NaN |
| 50 | W-11 | 22.96 | NaN |
| 51 | W-14 | 23.93 | NaN |
| 52 | W-15 | 34.02 | NaN |
| 53 | W-20 | 25.91 | NaN |
| 54 | W-25 | 31.46 | NaN |
| 55 | W-32 | 24.17 | NaN |
| 56 | W-34 | 20.02 | NaN |
| 57 | W-35 | 28.89 | NaN |
| 58 | W-36 | 22.26 | NaN |
| 59 | W-38 | 26.64 | NaN |

Table 2 shows PCR testing results for 149 positive COVID19 samples from Mar. 16, 2021 using wild type primer sets targeting the N1 region and a variant specific primer set targeting the variant specific (B.1.1.7, P.1, and B.1.351) mutations to the ORF1a region. The single Orf-mut primer set was able to successfully differentiate between wild type SARS-CoV-2 and any of the three B.1.1.7, P.1, and B.1.351 variants, producing a threshold qPCR signal only in the presence of those variants. Of note, primers for the detection of B.1.429/427 were not used in this experiment. The three detected variants (B.1.1.7, P.1, and B.1.351) were found in about 52.2% (72/138) of tested SARS-CoV-2 positive patients between March 16 and April 7, commiserate with the variant infection rates observed in the general population.

TABLE 2

| | | | | VOC |
|---|---|---|---|---|
| | | WT-primer | Mutant-primer | (B.1.429/427 |
| Sample # | Pts ID# | (N1) | (Orf-mut) | not included) |
| 1 | 1A6 | 34.05 | NaN | no |
| 2 | 1C3 | 35.41 | 37.45 | no |
| 3 | 1C7 | 21.53 | NaN | no |
| 4 | 1C8 | 22.49 | NaN | no |
| 5 | 1E3 | 23.03 | 39.14 | no |
| 6 | 1F4 | 34.95 | NaN | no |
| 7 | 1G6 | 18.28 | 19.57 | yes |
| 8 | 1H1 | 28.14 | 29.34 | yes |
| 9 | 1H3 | 22.21 | 24.58 | yes |
| 10 | 2A5 | 26.86 | NaN | no |
| 11 | 2B1 | 23.13 | 24.68 | yes |
| 12 | 2B3 | 30.02 | NaN | no |
| 13 | 2B8 | 26.99 | 28.95 | yes |
| 14 | 2B9 | 17.99 | 19.87 | yes |
| 15 | 2D5 | 28.61 | 30.09 | yes |
| 16 | 3B4 | 35.56 | NaN | no |
| 17 | 3D2 | 30.15 | 31.07 | yes |
| 18 | 4A12 | 18.08 | 35.29 | no |
| 19 | 4B8 | 25.57 | NaN | no |
| 20 | 4C10 | 27.65 | 29.02 | yes |
| 21 | 4C2 | 14.91 | 16.78 | yes |
| 22 | 4C7 | 32.04 | NaN | no |
| 23 | 4G3 | 33.84 | NaN | no |
| 24 | 4G4 | 23.97 | 25.92 | yes |
| 25 | 5C3 | 19.35 | NaN | no |
| 26 | 5D2 | 36.4 | N/A | N/A |
| 27 | 5G3 | 22.39 | 22.59 | yes |
| 28 | 6C11 | 19.40 | 16.87 | yes |
| 29 | 6D4 | NaN | NaN | N/A |
| 30 | 6E7 | NaN | NaN | N/A |
| 31 | 6E8 | 21.71 | 22.39 | yes |
| 32 | 6F10 | NaN | NaN | N/A |
| 33 | 6F11 | NaN | NaN | N/A |
| 34 | 0323-1-B1 | 33.65 | NaN | no |
| 35 | 0323-1-C5 | 22.37 | NaN | no |
| 36 | 0323-1-D1 | 26.13 | NaN | no |
| 37 | 0323-1-D6 | 29.25 | 31.56 | yes |
| 38 | 0323-1-E2 | 21.33 | 23.13 | yes |
| 39 | 0323-1-E6 | 35.86 | NaN | no |
| 40 | 0323-1-F4 | 21.96 | 39.92 | no |
| 41 | 0323-1-F5 | 25.72 | NaN | no |
| 42 | 0323-1-F6 | 17.85 | NaN | no |
| 43 | 0323-2A2 | 16.85 | 19.07 | yes |
| 44 | 0323-2B9 | 10.58 | NaN | no |
| 45 | 0323-2D12 | 23.02 | NaN | no |
| 46 | 0323-2E10 | 29.43 | NaN | no |
| 47 | 0323-2F10 | 22.53 | 38.83 | no |
| 48 | 0323-3B6 | NaN | 32.47 | yes |
| 49 | 0324-1C12 | 24.36 | 25.69 | Yes |
| 50 | 0324-1E1 | 32.39 | 28.99 | yes |
| 51 | 0324-1G1 | 33.69 | NaN | no |
| 52 | 0324-1G4 | 31.4 | 33.39 | yes |
| 53 | 0324-2F2 | 28.53 | 30.33 | yes |
| 54 | 0325-1A9 | 21.22 | NaN | no |
| 55 | 0325-1C4 | 32.1 | NaN | no |
| 56 | 0325-1D3 | 22.38 | 38.86 | no |
| 57 | 0325-1E3 | 18.17 | 23.31 | yes |
| 58 | 0325-1F12 | 18.77 | 19.75 | yes |
| 59 | 0325-1G1 | 36.53 | NaN | N/A |
| 60 | 0325-1G5 | 35.09 | NaN | no |
| 61 | 0325-2B1 | 28.51 | NaN | no |
| 62 | 0325-2G5 | 12.84 | 15.43 | yes |
| 63 | 0326-1B2 | 19.96 | 37.8 | no |
| 64 | 0326-1C1 | 22 | 23.68 | yes |
| 65 | 0326-1C5 | 23.57 | 25.56 | yes |
| 66 | 0326-2E6 | | | N/A |
| 67 | 0326-2F7 | 16.89 | 33.38 | no |
| 68 | 0327-1D3 | 22.42 | 24.24 | yes |
| 69 | 0327-1F2 | 21.66 | 23.19 | yes |
| 70 | 0327-1G6 | 33.77 | NaN | no |
| 71 | 0327-2D10 | 23.18 | 25.13 | yes |
| 72 | 0327-2D12 | 15.62 | 34.92 | no |
| 73 | 0330-1A7 | 30.68 | 32.15 | yes |
| 74 | 0330-1A8 | 34.52 | NaN | no |
| 75 | 0330-1B9 | 33.36 | NaN | no |
| 76 | 0330-1B10 | 21.84 | 23.46 | yes |
| 77 | 0330-1C12 | 27.11 | NaN | no |
| 78 | 0330-1D7 | 26.18 | NaN | no |
| 79 | 0330-1E10 | 14.08 | 17.13 | yes |
| 80 | 0330-1F11 | 35.48 | 36.90 | no |
| 81 | 0330-2G11 | 20.73 | 38.46 | no |
| 82 | 0330-3E5 | 26.34 | 25.97 | yes |
| 83 | 0331-1B5 | 12.69 | 18.33 | yes |
| 84 | 0331-1C2 | 32.33 | 32.88 | yes |
| 85 | 0331-1E6 | 27.51 | 31.15 | yes |
| 86 | 0401-1A2 | 19.87 | 22.64 | yes |
| 87 | 0401-1A10 | 25.97 | NaN | no |
| 88 | 0401-1B11 | 19.87 | 23.01 | yes |
| 89 | 0401-1C8 | 30.16 | 32.02 | yes |
| 90 | 0401-1D5 | 29.97 | 30.76 | yes |
| 91 | 0401-1E1 | 24.22 | 27.43 | yes |
| 92 | 0401-1E2 | 33.07 | 35.03 | yes |
| 93 | 0401-1F1 | 20.95 | 36.77 | no |
| 94 | 0401-2A7 | 35.13 | 38.09 | no |
| 95 | 0401-2C5 | 23.19 | 27.97 | yes |
| 96 | 0401-2D7 | 16.35 | 19.10 | yes |
| 97 | 0401-2D10 | 15.08 | 17.89 | yes |
| 98 | 0401-2F11 | NaN | NaN | N/A |
| 99 | 0401-2H3 | 14.12 | 36.23 | no |
| 100 | 0402-1B2 | 28.8 | 31.75 | yes |
| 101 | 0402-1B5 | 26.76 | 38.23 | no |
| 102 | 0402-1C3 | 25.47 | 25.96 | yes |
| 103 | 0402-1D4 | 17.35 | 19.01 | yes |
| 104 | 0402-1D6 | 32.44 | 33.33 | yes |
| 105 | 0402-1E6 | 31.35 | NaN | no |
| 106 | 0402-1H3 | 27.23 | 29.71 | yes |
| 107 | 0402-1H6 | 32.38 | 34.17 | yes |
| 108 | 0402-2B4 | 19.81 | NaN | no |
| 109 | 0403-1A1 | 29.03 | NaN | no |
| 110 | 0403-1A6 | 33.29 | 35.93 | yes |
| 111 | 0403-1B2 | 26.4 | 28.51 | yes |
| 112 | 0403-1C3 | 34.73 | 36.35 | no |
| 113 | 0403-1C5 | 24.57 | 28.25 | yes |
| 114 | 0403-1D6 | 33.36 | NaN | no |
| 115 | 0403-1F1 | 33.93 | NaN | no |
| 116 | 0403-1F2 | NaN | NaN | N/A |
| 117 | 0403-1F6 | 16.07 | 22.25 | yes |
| 118 | 0403-1H4 | 36.73 | NaN | N/A |
| 119 | 0403-1H6 | 25.77 | NaN | no |
| 120 | 0403-2B3 | 17.47 | 38.74 | no |
| 121 | 0403-2C12 | 29.24 | 32.07 | yes |
| 122 | 0403-2F9 | 18.1 | 36.47 | no |
| 123 | 0403-2G2 | NaN | NaN | N/A |
| 124 | 0406-1C5 | 25.84 | 27.54 | yes |
| 125 | 0406-1D6 | 24.17 | 25.19 | yes |
| 126 | 0406-1E5 | 25.03 | 26.52 | yes |
| 127 | 0406-1E7 | 28.99 | 29.61 | yes |
| 128 | 0406-1G3 | 17.22 | 19.83 | yes |
| 129 | 0406-2B1 | 31.9 | 34.9 | yes |
| 130 | 0406-2B2 | 28.84 | 30.42 | yes |
| 131 | 0406-2G1 | NaN | NaN | N/A |
| 132 | 0406-2G2 | 28.28 | 29.61 | yes |
| 133 | 0406-2H1 | 31.74 | 33.01 | yes |
| 134 | 0406-3G3 | 31.32 | NaN | no |
| 135 | 0406-3H3 | 33.77 | 34.19 | yes |
| 136 | 0406-2A7 | 21.72 | 37.56 | no |
| 137 | 0406-2B8 | 21.82 | 23.75 | yes |
| 138 | 0406-2B9 | 29.25 | 30.63 | yes |
| 139 | 0407-1-B06 | 25.32 | 28.06 | yes |
| 140 | 0407-1-C02 | 23.55 | 24.97 | yes |
| 141 | 0407-1-C10 | 24.03 | NaN | no |
| 142 | 0407-1-F09 | 35.86 | NaN | no |
| 143 | 0407-1-H03 | 36.90 | NaN | no |
| 144 | 0407-2-D12 | NaN | NaN | no |
| 145 | 0407-2-E04 | 28.66 | 29.81 | yes |
| 146 | 0407-2-E10 | 26.37 | NaN | no |
| 147 | 0407-2-F10 | 33.38 | NaN | no |
| 148 | 0407-2-G12 | 17.27 | 37.03 | no |
| 149 | 0407-2-H07 | 19.62 | NaN | no |

To validate the rapid PCR detection of SARS-CoV-2 variants using methods and compositions of the invention, samples were tested with primer sets of the invention targeting mutations in the ORF1a region and the S region (Orf-mut primers and S1-mut primers). The lineage of the SARS-CoV-2 infections were then confirmed by next generation sequencing (NGS). The results are shown in Table 3.

For the first batch, 30 samples were used made up of 20 SARS-CoV-2 positive paired nasal-saliva specimens collected at 2 time points (10 pairs) and 10 SARS-CoV-2 negative (saliva) samples. Samples were collected with DNA Genotek devices (OR-100 for nasal swabs and OM-505 for saliva; current device names ORE-100 and OME-505). The samples were collected from 2020 Nov. 12 to 2021 Jan. 10. No VOC were detected (except B.1.429/427 not included).

A second batch of 11 SARS-CoV-2 positive (7 by rapid saliva PCR test and 4 by rapid nasal swab PCR test) and 9 SARS-CoV-2 negative (6 by rapid saliva PCR test and 3 by rapid nasal swab PCR test) samples was tested. The samples were collected from 2021 Nov. 12 until 2021 Feb. 18. No VOC (except B.1.429/427) was detected.

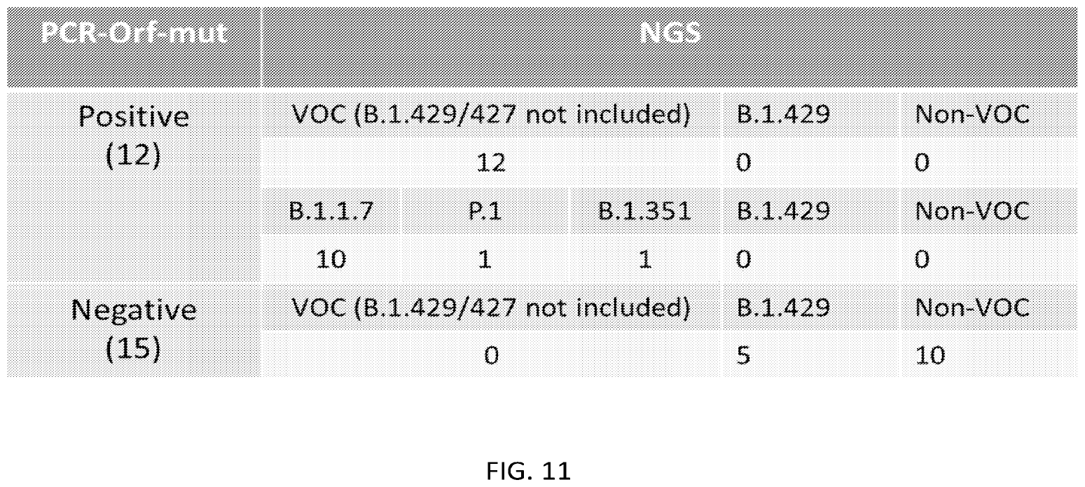

A third batch of 33 SARS-CoV-2 positive samples (25 by rapid saliva PCR test and 8 by rapid nasal swab PCR test) was tested. In this batch, 28 had a lineage designation of one of the VOC that matched Orf-mut PCR result. Of 12 Orf-mut PCR positive cases, ten were B.1.1.7, one was P1, and one was B.1.351. Of the 14 Orf-mut PCR negative cases, none of them belonged to the lineage of B.1.1.7, P1, or B.1.351. The results are shown in Table 3 below and FIG. 11. Using a single Orf-mut primer set of the invention, detection of the three targeted VOC (B.1.1.7, P1, or B.1.351) was in 100% positive and 100% negative agreement with the NGS results, validating the detection methods of the invention.

TABLE 3

| SARS-Cov2 positive | Orf-mut | S1-mut | Lineage |
|---|---|---|---|
| samples | (PCR) | (PCR) | (NGS) |
| SA-1 | No | N/A | USA-B.1.2 |
| SA-5 | No | N/A | california-B.1.429 |
| SA-7 | No | N/A | USA-B.1.2 |
| SA-8 | No | N/A | USA-B.1.2 |
| SW-1 | No | N/A | california-B.1.429 |
| SW-4 | No | N/A | B.1.596 |
| 1-C7 | No | N/A | california-B.1.429 |
| 1-C8 | No | N/A | california-B.1.429 |
| 1-E3 | No | N/A | USA-B.1.2 |
| 1-G6 | yes | yes | B.1.1.7 |
| 1-H3 | yes | yes | B.1.1.7 |
| 2-A5 | No | N/A | USA-B.1.596 |
| 2-B1 | yes | yes | B.1.1.7 |
| 2-B8 | yes | yes | B.1.1.7 |
| 2-D5 | yes | yes | B.1.1.7 |
| 4-A12 | No | N/A | USA-B.1.2 |
| 4-B8 | No | N/A | california-B.1.429 |
| 4-C10 | yes | yes | B.1.1.7 |
| 4-C2 | yes | yes | B.1.1.7 |
| 4-C7 | No | N/A | USA/mexico-B.1.609 |
| 4-G4 | yes | no | Brazil-P1 |
| 5-C3 | No | N/A | USA/mexico-B.1.1.519 |
| 5-G3 | yes | yes | B.1.1.7 |
| 6-C11 | yes | yes | B.1.1.7 |
| 6-D4 | yes | no | South Africa-B.1.351 |
| 6-E8 | yes | yes | B.1.1.7 |
| 6-F10 | No | N/A | USA-B.1.596 |

### Example 3—Saliva and Nasal Swab Examples

The following provides exemplary protocols for detection of viral nucleic acid in accordance with methods of the present invention. A biological sample is obtained and may include a human bodily fluid and may be collected in any clinically acceptable manner.

For many respiratory infections, a biological sample is generally collected via a nasal or throat swab, or, in some cases, saliva. In other examples, the sample may include an aerosol sample or droplets obtained in air or, more preferably, via the expulsion of droplets with a cough or sneeze.

### On-Site Saliva Sample Collection:

Saliva samples may be collected from individuals by, for example, having them spit into a provided sterile container. Saliva collection devices may include, for example, a Nest 1.9 ml cryogenic vial (or “Nest tube”) with screw cap (externally threaded) with a pre-printed 10-digit one-dimension barcode on the side and a laser-etched DATAMATRIX two-dimension code at the bottom will be used as the container of the saliva sample. A saliva collection support funnel (Nest) will be used in tandem with the Nest vial.

### Saliva Sample Receiving and Accessioning in Lab:

Samples are transported to the lab. Samples are removed from bags and visually examined by the accessioning supervisor at the receipt desk for any leakage or damage. Samples passed the pre-screening step by the supervisor are moved to the desktop used by the accessioning team. Samples failed the pre-screening step are set aside for further investigation. Accessioners will scan the barcodes on the Nest tubes and examine patient information and consent status shown on a computer screen via a laboratory information management system (LIMS). Tubes with complete patient information in the LIMS and have no leakage (i.e., qualified samples), are placed in a bar-coded 48-format rack. The positions of the samples in the rack should match assigned positions in the LIMS. Disqualified samples are placed in another bar-coded 48-format rack and set aside for further investigation by the accessioning supervisor. The rack of samples may then be placed on a platform rocker in hold position @ 60 rpm until a medical lab scientist (MLS) from the sample preparation team fetches the samples.

### Saliva Reaction Buffer:

As part of sample preparation, the saliva sample is mixed with a unique buffer composition prepared specifically for saliva (referred to herein as Saliva Preparation Buffer). Preparation of the Saliva Preparation Buffer includes use of at least the following equipment: Biosafety cabinet or laminar flow hood (workspace capable of maintaining an aseptic environment); individual, sterile wrapped pipettes, pipette tips, such as 10 and 25 mL; pipette aid; pipettor, 1 mL or 200 μL and corresponding tips; and 50 ml sterile, nuclease-free Falcon tubes. An exemplary Saliva Preparation Buffer comprises the following reagents/components:
0.5 M Bond-Breaker TCEP solution, (Tris(2-carboxyethyl)phosphine hydrochloride, neutral pH), Sterile, DNase-, RNase- and Protease-Free grade, ThermoFisher Scientific, catalog number 77720, 5 mL;
RNase inhibitor, human placenta, 40,000 units/ml, Sterile, DNase-, RNase-Free grade, New England Biolabs, catalog number M0307L, 10,000 units, 250 ul/tube;
Amphotericin B solution, 250 g/ml in deionized water, sterile, Sigma-Aldrich, catalog number A2942, 100 ml (or similar antifungal at an appropriate concentration to prevent fungal contamination and growth);
Penicillin-Streptomycin Solution, 100×, a mix of Penicillin (10,000 IU) and Streptomycin (10,000 μg/ml) in a 100-fold working concentration, Sterile, Corning, catalog number 30-002-CI (or similar antibiotics at an appropriate concentration to prevent bacteria contamination and growth;
Nuclease-free water, Sterile, Millipore/Sigma, W4502, DNase-, RNase- and Protease-Free grade; and
Disinfectant, such as 70% ethanol.

Preparation the Saliva Preparation Buffer is in accordance with standard biological and/or clinical laboratory practices and procedures and is performed in a biosafety cabinet or laminar flow hood.

Preparation of the ingredients includes at least the following steps: clean work surface with appropriate disinfectant; disinfect reagent bottles prior to placing on work surface; aliquot nuclease-free water, 40 mL in 50 mL sterile Falcon tube, store at RT; aliquot Amphotericin B 4 ml/tube (in 5 ml sterile corning tube), store at −20 C; aliquot Penicillin/Streptomycin, 1 ml/tube (in sterile Eppendorf tubes), store at −20 C; and record lot information and preparation in a laboratory-controlled notebook.

Preparation of the Saliva Preparation Buffer includes at least the following steps:
1. Clean work surface with appropriate disinfectant;
2. Disinfect reagent bottles (aliquot, except RNase inhibitor) prior to placing on work surface;
3. For example, to prepare 5 mL buffer (for 1000 tests):
   3.1. in a 15 mL sterile falcon tube, add 4.3 mL nuclease-free water;
   3.2. add 400 uL TCEP;
   3.3. using a sterile pipette, add 50 ul of RNase inhibitor;
   3.4 thaw a tube of Amphotericin and a tube of Penicillin/Streptomycin, using a sterile pipette, aseptically add 200 uL of Amphotericin and 50 uL of Penicillin/Streptomycin to the 15 mL falcon tube;
4. Record lot information and preparation in a laboratory-controlled notebook;
5. Assign laboratory appropriate identification (e.g. lot number);
6. Cap the tube securely and mix thoroughly by inverting the tube;
7. Withdraw 100 ul of medium for QC sample;
8. Label the bottle as:
   Saliva Reaction BUFFER
   Lab ID: (Insert laboratory appropriate identification, such as STB1 as Summit Buffer 1)
   DOM: (Insert current date of manufacture)
   Expires: (Insert date 1 month after manufacture date)
   Store at 2-8 C
9. Store at 2-8 C, add 5 ul/each test together with 30 uL saliva and 5 uL proteinase K when performing salivaFAST testing; and
10. Perform sterility check.

### Saliva Sample Preparation:

An MLS from the sample preparation team will fetch the racks of accessioned samples on the rocker and bring them into the sample preparation room to prepare them for testing. They will bring prepared 96-well Sample Prep Plate (SPP) containing 10 μL/well of a Sample Prep Mix (SPM). The SPM contains the Saliva Preparation Buffer and a protease (Proteinase K). In particular, the 96-well SPP contains 10 μL SPM (5 μL Saliva Preparation Buffer and 5 μL Proteinase K (Promega))/well, dispensed into each well using a multichannel equalizer or Viaflow (Integra). Samples are decapped with a semi-automated 6-channel decapper (Brooks) or automated 48-format decapper (Brooks) inside the biosafety cabinets. Caps will be temporarily placed on the cap carrier rack when using the 6-channel decapper. Approximately 30 μL of saliva are transferred from the tubes in the 48-well rack using the E1-ClipTip electronic multichannel (8-channels) equalizer to the 96-well SPP containing the 10 μL SPM and pipetting well. Two 48-well racks of samples will fill one 96-well SPP. Samples are recapped (6 at a time if using the 6-channel decapper or 48 at a time if using the automated 48-format decapper). The saliva and SPM are mixed well by placing the plates on the digital microplate shaker @ 500 RPM for 1 minute. The plate is placed on the miniAmp 96-well PCR instrument at 95° C. for 5 minutes, and 4° C. on hold. The entire racks of samples are then brought to the temporary sample storage area. Any of the samples that require repeat testing will be identified from the temporary sample storage area. Repeat testing is only allowed one time. If failed, request a new sample. Store left-over samples in −80° C. for future use.

### PCR Reagent Preparation and Plate Setup (Saliva Testing):

A plate containing a PCR master mix (herein referred to as a PCR Master Mix Plate (PMMP), includes 12.5 μL of PCR master mix dispensed into each well of the plate using a multichannel equalizer or Viaflow (Integra) on to a 96- or 384-well plate. The PCR master mix is composed of 10 μL Luna Universal Probe One-Step Reaction Mix, 1 μL Luna Warmstart RT enzyme Mix, and 1.5 μL of a primer/probe set. The 1.5 μL of primer/probe set will be made as: 6.7 M working stocks of any of the VOC(s)-specific primer, the N primers, and/or RNP primers and 1.7 μM FAM-labeled N1 probe, ATTO-647 labeled RNP probe, or one of the VOC-specific labeled probes by adding 50.25 μL of each 100 μM primers and probe stock to 524 μL IDTE buffer (pH7.5). Exemplary VOC-specific primers and probes are listed in FIG. 1 and above.

The MLS in the molecular team will place a 96- or a 384-well PMMP into their individual PCR workstation and add 7.5 μL of treated saliva sample from the Saliva Sample Preparation Step to each designated well of the PMMP. The treated saliva sample is then mixed with the PCR master mix by pipetting, taking care to avoid introducing bubbles. The MLS can then add 7.5 μL of positive control (IDT synthetic 2019-SARS-CoV-N control, 4000 copies/uL or one or more VOC-positive controls), and negative control (IDT Hs-RPP30 control, 4000 copies/μL) for SARS-CoV-2 and/or one or more VOCs, and no-template control (NTC-water) to designated PCR wells for the controls (1 positive control, 1 negative control, and NTC per plate) and mixes by pipetting, avoiding introducing bubbles. The MLS then places a transparent plastic qPCR film on the PMMP and seals the film with a plate sealer and spin briefly to remove bubbles with a plate spinner.

### PCR Thermal Profile (Amplification Area) (Saliva Testing):

Load the plate into a Bio-Rad CFX or a QuantStudio PCR machine, Open master file “ST-CoV-PCR protocol”, and run the following thermocycler conditions:
1. Step 1:55° C. 10 minutes, 1 cycle;
2. Step 2:95° C. 1 minute, 1 cycle; and
Step 3:95° C. 10 sec, 60° C. 30 sec (+plate read at both FAM channel for N1 target & Cy5 channel for RNP target) for 40 cycles.

### Data Interpretation (BioRad CFX Opus 96-Well Format) (Saliva Testing):

The Bio-Rad CFX reports Cq values, in which the Cq value files (csv file) are exported from the PCR machine to the OvDx LIMS. Interpretation of the Cq values (DETECTED, NOT DETECTED, and INVALID) will be exported to the OvDx LIMS. A Cq less than or equal to 36 is interpreted as positive for the target VOC (determined by the VOC-specific primer-probe set in that well)

### On-Site Nasal Swab (Anterior Nares) Sample Collection:

Nasal swab collection devices include: a 1.9 ml Nest tube filled with 1 ml a unique buffer composition specific to nasal swab samples (hereinafter referred to as Swab Transport Buffer), which will be used as the container of the nasal swab sample; and an oral/Nares swab by Nest will be used to swab the patient's anterior nares and later be placed inside the Nest tube filled with the Swab Transport Buffer.

The nasal swab (anterior nares) should be collected under the supervision of a trained healthcare worker designated by the organization overseeing the collection site. The healthcare worker supervising the collection should clean hands with alcohol-based sanitizer or fragrance-free soap and water and don appropriate PPE (gown, gloves, face mask, and/or face shield). Before collection, patients are provided instructional materials such as this one recommended by the FDA (https://tinyurl.com/nasalswab1-2). The healthcare worker ensures all patient information, including name, date of birth, and additional information required by state reporting rules, is filled out properly before collection. The healthcare worker then asks the patient to review a study consent form to opt in or out of the study (provided by Ovation). Lastly, the healthcare worker will scan a pre-printed barcode label to tie it to the patient information that is already collected, then place the label on the Nest tube that will be used by the patient.

The healthcare worker removes the cap of the Nest tube, directs the patient to swab their anterior nares ten times for each nares, and breaks the swab inside the tube at the proximal breakpoint. The healthcare worker will replace the cap of the Nest tube and make sure it's securely tightened. If there is any sample spill during the collection process, the healthcare worker will use an alcohol wipe or equivalent to wipe the outside of the tube to prevent contamination. The sample will then be placed in an individual bag under room temperature before being transported to the lab.

The healthcare worker supervising the swab sample collection should use alcohol-based hand sanitizer after handling each patient's sample.

### Swab Sample Receiving and Accessioning in Lab:

Samples are transported to the lab. Samples will be removed from bags and visually examined by the accessioning supervisor at the receipt desk for any leakage or damage. Samples passed the pre-screening step by the supervisor are moved to the desktop used by the accessioning team. Samples failed the pre-screening step are set aside for further investigation. Accessioners will scan the barcodes on the Nest tubes and examine patient information and consent status shown on a computer screen via a laboratory information management system (LIMS). Tubes with complete patient information in the LIMS and have no leakage (i.e., qualified samples), are placed in a rack. The positions of the samples in the rack should match assigned positions in the LIMS. Disqualified samples are placed in another rack and set aside for further investigation by the accessioning supervisor. The rack of samples may then be placed on a platform rocker in hold position @ 600 rpm until a medical lab scientist (MLS) from the sample preparation team fetches the samples.

### Swab Preparation Buffer:

As part of sample preparation, the swab sample will be mixed with a unique buffer composition prepared specifically for swab samples (referred to herein as Swab Preparation Buffer). Preparation of the Swab Preparation Buffer includes use of at least the following equipment: Biosafety cabinet or laminar flow hood (workspace capable of maintaining an aseptic environment); individual, sterile wrapped pipettes, pipette tips, such as 10 and 25 mL; pipette aid; pipettor, 1 mL or 200 μL and corresponding tips; 50 ml sterile, nuclease-free Falcon tubes; Eppendorf repeater (50 mL capacity); 1.9 ml Cryovial tubes, Nest; Nest tube racks; and screw cap tube decapper equipment, Brooks Life Sciences.

The preparation of the Swab Transport Buffer further includes use of at least the following reagents/components:
10×TBE Buffer (Tris-Borate-EDTA, pH 8.2-8.4), Sterile, DNase-, RNase- and Protease-Free grade, Fisher BioReagents, catalog number BP133320, 20L;
RNase inhibitor, human placenta, 40,000 units/ml, Sterile, DNase-, RNase-Free grade, New England Biolabs, catalog number M0307L, 10,000 units, 250 ul/tube;
Amphotericin B solution, 250 μg/ml in deionized water, sterile, Sigma-Aldrich, catalog number A2942, 100 ml (or similar antifungal at an appropriate concentration to prevent fungal contamination and growth);
Penicillin-Streptomycin Solution, 100×, a mix of Penicillin (10,000 IU) and Streptomycin (10,000 μg/ml) in a 100-fold working concentration, Sterile, Corning, catalog number 30-002-CI (or similar antibiotics at an appropriate concentration to prevent bacteria contamination and growth;
Nuclease-free water, Sterile, Millipore/Sigma, W4502, DNase-, RNase- and Protease-Free grade; and
Disinfectant, such as 70% ethanol.

Preparation of the ingredients includes at least the following steps: clean work surface with appropriate disinfectant; disinfect reagent bottles prior to placing on work surface; aliquot 10×TBE Buffer, 500 ml/bottle in Corning 500 ml sterile bottle, store at RT; aliquot nuclease-free water, 894.95 ml/bottle in Corning 1 L sterile bottle, store at RT; aliquot Amphotericin B solution 4 ml/tube (in 5 ml sterile Corning tube), store at −20 C; aliquot Penicillin/Streptomycin, 1 ml/tube (in sterile Eppendorf tubes), store at −20 C; and Record lot information and preparation in a laboratory-controlled notebook.

Preparation of the Swab Preparation Buffer includes at least the following steps:
1. Clean work surface with appropriate disinfectant;
2. Disinfect reagent bottles (aliquot, except RNase inhibitor) prior to placing on work surface;
3. For example, to prepare 1 L viral transport buffer:
   3.1. bring 1 bottle of nuclease-free water (894.95 ml/bottle);
   3.2. using a sterile 50 ml falcon tube, add 100 ml of 10×TBE Buffer;
   3.3. using a sterile pipette, add 50 μl of RNase inhibitor; and
   3.4 thaw a tube of Amphotericin B solution and a tube of Penicillin/Streptomycin, using a sterile pipette, aseptically add 4 ml of Amphotericin and 1 ml of Penicillin/Streptomycin to the bottle.
4. Record lot information and preparation in a laboratory-controlled notebook;
5. Assign laboratory appropriate identification (e.g. lot number);
6. Cap the tube securely and mix thoroughly by inverting the tube;
7. Withdraw 100 ul of medium for QC sample;
8. Label the bottle as:
   SWAB TRANSPORT BUFFER
   Lab ID: (Insert laboratory appropriate identification, such as STB2 as Summit Buffer 2)
   DOM: (Insert current date of manufacture)
   Expires: (Insert date 1 month after manufacture date)
   Store at 2-8 C
9. Store at 2-8 C, until dispensed into aliquots;
10. Aliquot 1 mL of prepared Swab Preparation Buffer into individual sterile 1.9 ml screw-capped tubes (Nest) using Eppendorf repeater (50 mL capacity) and Brooks decapper;
11. Perform sterility check; and
12. Store tubes and any buffer remaining in the bottle at 2-8 C.

### Swab Sample Preparation:

An MLS from the sample preparation team will fetch the racks of accessioned samples on the rocker and bring them into the sample preparation room to prepare them for testing. They will bring prepared 96-well Sample Prep Plate (SPP) containing 5 μL/well of protease (Proteinase K). In particular, the 96-well SPP contains 5 μL of Proteinase K (Promega))/well, dispensed into each well using a multichannel equalizer or Viaflow (Integra). Samples are decapped with a semi-automated 6-channel decapper (Brooks) or automated 48-format decapper (Brooks) inside the biosafety cabinets. Caps will be temporarily placed on the cap carrier rack when using the 6-channel decapper. Approximately 35 μL of swab sample are transferred from the tubes in the 48-well rack using the E1-ClipTip electronic multichannel (8-channels) equalizer to the 96-well SPP containing the 5 μL of Proteinase K and pipetting well. Two 48-well racks of samples will fill one 96-well SPP. Samples are recapped (6 at a time if using the 6-channel decapper or 48 at a time if using the automated 48-format decapper). The swab samples and Proteinase K are mixed well by placing the plates on the digital microplate shaker @ 500 RPM for 1 minute. The plate is placed on the miniAmp 96-well PCR instrument at 95° C. for 5 minutes, and 4° C. on hold. The entire racks of samples are then brought to the temporary sample storage area. Any of the samples that require repeat testing will be identified from the temporary sample storage area. Repeat testing is only allowed one time. If failed, request a new sample. Store left-over samples in −80° C. for future use.

### PCR Reagent Preparation and Plate Setup (Swab Testing):

A plate containing a PCR master mix (herein referred to as a PCR Master Mix Plate (PMMP), includes 12.5 μL of PCR master mix dispensed into each well of the plate using a multichannel equalizer or Viaflow (Integra) on to a 96- or 384-well plate. The PCR master mix is composed of 10 μL Luna Universal Probe One-Step Reaction Mix, 1 μL Luna Warmstart RT enzyme Mix, and 1.5 μL of primer/probe set for wild type or VOC SARS-CoV-2 detection. The 1.5 μL N1/RNP primer/probe will be made as: 6.7 μM working stocks of the primers and 1.7 μM of the labeled probe associated with that primer by adding 50.25 μL of each 100 μM primers and probe stock to 524 μL IDTE buffer (pH7.5).

The MLS in the molecular team will place a 96- or a 384-well PMMP into their individual PCR workstation and add 7.5 μL of treated swab sample from the Swab Sample Preparation Step to each designated well of the PMMP. The treated swab sample is then mixed with the PCR master mix by pipetting, taking care to avoid introducing bubbles. The MLS then adds 7.5 μL of a positive control (IDT synthetic 2019-SARS-CoV-N control, 4000 copies/uL or one or more VOC-positive control), and negative control (IDT Hs-RPP30 control, 4000 copies/μL) for SARS-CoV-2 or a VOC thereof, and no-template control (NTC-water) to designated PCR wells for the controls (1 positive control, 1 negative control, and NTC per plate) and mixes by pipetting, avoiding introducing bubbles. The MLS then places a transparent plastic qPCR film on the PMMP and seals the film with a plate sealer and spin briefly to remove bubbles with a plate spinner.

### PCR Thermal Profile (Amplification Area) (Swab Testing):

Load the plate into a Bio-Rad CFX or a QuantStudio PCR machine, Open master file “ST-CoV-PCR protocol”, and run the following thermocycler conditions:
1. Step 1:55° C. 10 minutes, 1 cycle;
2. Step 2:95° C. 1 minute, 1 cycle; and
Step 3:95° C. 10 sec, 60° C. 30 sec (+plate read at both FAM channel for N1 target & Cy5 channel for RNP target) for 40 cycles.

### Data Interpretation (BioRad CFX Opus 96-Well Format) (Swab Testing);

The Bio-Rad CFX reports Cq values, in which the Cq value files (csv file) are exported from the PCR machine to the OvDx LIMS. Interpretation of the Cq values (DETECTED, NOT DETECTED, and INVALID) will be exported to the OvDx LIMS. A Cq less than or equal to 36 is interpreted as positive for the target VOC (determined by the VOC-specific primer-probe set in that well)

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for detecting SARS-CoV-2 variants, the method comprising:
obtaining a biological sample comprising nucleic acid;
amplifying the nucleic acid with primers specific to one or more target SARS-CoV-2 variants without amplification of wild type SARS-CoV-2 nucleic acid; and
analyzing amplicons produced in the amplifying step to detect the presence of the one or more target SARS-CoV-2 variants.

2. The method of claim 2 further comprising detecting the presence of a SARS-CoV-2 infection by, prior to the amplifying step using primers that amplify the one or more target SARS-CoV-2 variants and wild type SARS-CoV-2.

3. The method of claim 1 wherein the one or more target SARS-CoV-2 variants are selected from the group consisting of B.1.1.7, P.1, B.1.351, and B.1.429/427.

4.-6. (canceled)

7. The method of claim 3 wherein the primers target an ORF-1A region.

8. (canceled)

9. The method of claim 7 wherein the primers comprise SEQ ID NO: 1 and SEQ ID NO: 2.

10. The method of claim 9 wherein the amplifying step comprises quantitative PCR (qPCR) using a probe comprising SEQ ID NO: 3.

11.-12. (canceled)

13. The method of claim 3 wherein the primers comprise SEQ ID NO: 4 and SEQ ID NO: 5.

14. The method of claim 13 wherein the amplifying step comprises quantitative PCR (qPCR) using a probe comprising SEQ ID NO: 6.

15. The method of claim 3 wherein the one or more target SARS-CoV-2 variants comprise B.1.351 and the primers target a G25563T or G28887T substitution relative to wild type SARS-CoV-2.

16. The method of claim 15 wherein the primers comprise SEQ ID NO: 19 and SEQ ID NO: 20 or SEQ ID NO: 22 and SEQ ID NO: 23.

17. The method of claim 16 wherein the amplifying step comprises quantitative PCR (qPCR) using a probe comprising SEQ ID NO: 21 or SEQ ID NO: 24.

18. The method of claim 3 wherein the one or more target SARS-CoV-2 variants comprise B.1.429/427 and the primers target G27890T/G27987T or G28191T/A28272T substitutions relative to wild type SARS-CoV-2.

19. The method of claim 18 wherein the primers comprise SEQ ID NO: 25 and SEQ ID NO: 26 or SEQ ID NO: 28 and SEQ ID NO: 29.

20. The method of claim 19 wherein the amplifying step comprises quantitative PCR (qPCR) using a probe comprising SEQ ID NO: 27 or SEQ ID NO: 30.

21. The method of claim 1 further comprising:
mixing the biological sample in a buffer composition comprising nuclease-free water, an antifungal, an antibiotic, a ribonuclease inhibitor, and a reducing agent, wherein the amplifying step is performed on the nucleic acid in the buffer without prior extraction of the nucleic acid.

22.-23. (canceled)

24. The method of claim 21 wherein the reducing agent is a Tris(2-carboxyethyl)phosphine hydrochloride solution.

25. The method of claim 21 wherein the antifungal comprises an Amphotericin Band the antibiotic comprises Penicillin Streptomycin.

26. (canceled)

27. The method of claim 21 further comprising the step of heat inactivating the biological sample mixed with the buffer composition prior to said amplifying step.

28. The method of claim 27 wherein the mixture of biological sample and buffer composition is heated to 95° C. for 5 minutes.

29. The method of claim 1 wherein the biological sample is from a subject suspected of having a SARS-CoV-2 infection, the method further comprising selecting a treatment regimen based on the detected presence of the one or more target SARS-CoV-2 variants.

30.-50. (canceled)


SEQUENCE LISTING (33 sequences)

SEQ ID NO: 1 - DNA, 25 bp, Artificial Sequence
  Synthesized
atggttgatactagtttgaagctaa

SEQ ID NO: 2 - DNA, 22 bp, Artificial Sequence
  Synthesized
agcaccatcatcatacacagtt

SEQ ID NO: 3 - DNA, 30 bp, Artificial Sequence
  Synthesized
actgtgttatgtatgcatcagctgtagtgt

SEQ ID NO: 4 - DNA, 23 bp, Artificial Sequence
  Synthesized
tacttggttccatgctatatctg

SEQ ID NO: 5 - DNA, 24 bp, Artificial Sequence
  Synthesized
caccatcattaaatggtaggacag

SEQ ID NO: 6 - DNA, 26 bp, Artificial Sequence
  Synthesized
tcaaacctcttagtaccattggtccc

SEQ ID NO: 7 - DNA, 24 bp, Artificial Sequence
  Synthesized
atgatccatttttgggtgtttacc

SEQ ID NO: 8 - DNA, 24 bp, Artificial Sequence
  Synthesized
gcaattattcgcactagaataaac

SEQ ID NO: 9 - DNA, 25 bp, Artificial Sequence
  Synthesized
aagttggatggaaagtgagttcaga

SEQ ID NO: 10 - DNA, 24 bp, Artificial Sequence
  Synthesized
atgtctctaaatggaccccaaaat

SEQ ID NO: 11 - DNA, 24 bp, Artificial Sequence
  Synthesized
tctggttactgccagttgaatctg

SEQ ID NO: 12 - DNA, 24 bp, Artificial Sequence
  Synthesized
accccgcattacgtttggtggacc

SEQ ID NO: 13 - DNA, 24 bp, Artificial Sequence
  Synthesized
tcttgattctaaggttggtggtaa

SEQ ID NO: 14 - DNA, 23 bp, Artificial Sequence
  Synthesized
aaacctttaacaccattacaagg

SEQ ID NO: 15 - DNA, 26 bp, Artificial Sequence
  Synthesized
actgaaatctatcaggccggtagcac

SEQ ID NO: 16 - DNA, 22 bp, Artificial Sequence
  Synthesized
cacaccttgtaatggtgttaaa

SEQ ID NO: 17 - DNA, 25 bp, Artificial Sequence
  Synthesized
actactactctgtatggttggtaac

SEQ ID NO: 18 - DNA, 24 bp, Artificial Sequence
  Synthesized
tggtttccaacccacttatggtgt

SEQ ID NO: 19 - DNA, 19 bp, Artificial Sequence
  Synthesized
attgttggcgttgcacttc

SEQ ID NO: 20 - DNA, 22 bp, Artificial Sequence
  Synthesized
caacagcaagttgcaaacaaag

SEQ ID NO: 21 - DNA, 22 bp, Artificial Sequence
  Synthesized
catagcgcttccaaaatcataa

SEQ ID NO: 22 - DNA, 20 bp, Artificial Sequence
  Synthesized
tcatcacgtagtcgcaacag

SEQ ID NO: 23 - DNA, 20 bp, Artificial Sequence
  Synthesized
caaagcaagagcagcatcac

SEQ ID NO: 24 - DNA, 25 bp, Artificial Sequence
  Synthesized
gaatttctcctgctagaatggctgg

SEQ ID NO: 25 - DNA, 21 bp, Artificial Sequence
  Synthesized
tcacgcctaaactaacatgaa

SEQ ID NO: 26 - DNA, 21 bp, Artificial Sequence
  Synthesized
ggacacgggtcatcaactaga

SEQ ID NO: 27 - DNA, 28 bp, Artificial Sequence
  Synthesized
tcacaactgtagctgcatttcaccaaga

SEQ ID NO: 28 - DNA, 23 bp, Artificial Sequence
  Synthesized
cctaaattgggtagtcttgtatt

SEQ ID NO: 29 - DNA, 22 bp, Artificial Sequence
  Synthesized
ggtccattatcagacattatag

SEQ ID NO: 30 - DNA, 25 bp, Artificial Sequence
  Synthesized
agagtatcatgacgttcgtgttgtt

SEQ ID NO: 31 - DNA, 20 bp, Artificial Sequence
  Synthesized
tggtaattataattaccggt

SEQ ID NO: 32 - DNA, 26 bp, Artificial Sequence
  Synthesized
actgaaatctatcaggccggtagcac

SEQ ID NO: 33 - DNA, 23 bp, Artificial Sequence
  Synthesized
aaaccttcaacaccattacaagg